(19) United States
(12) Patent Application Publication
(10) Pub. No.: US 2015/0201889 A1
(43) Pub. Date: Jul. 23, 2015

(54) SONIFICATION OF IMAGING DATA

(71) Applicant: New York University, New York, NY (US)

(72) Inventors: Agnieszka Roginska, New York, NY (US); Kent Friedman, New York, NY (US); Hariharan Mohanraj, New York, NY (US)

(73) Assignee: NEW YORK UNIVERSITY, New York, NY (US)

(21) Appl. No.: 14/569,425

(22) Filed: Dec. 12, 2014

Related U.S. Application Data
(60) Provisional application No. 61/916,006, filed on Dec. 13, 2013.

(51) Int. Cl. A61B 5/00; A61B 19/00

(52) U.S. Cl. CPC A61B 5/7415; A61B 19/5225; A61B 5/4064; A61B 2576/026; A61B 2019/5236; A61B 2019/524

(57) ABSTRACT

Scans of brains result in data that can be challenging to display due to its complexity, multi-dimensionality, and range. Visual representations of such data are limited due to the nature of the display, the number of possible dimensions that can be represented visually, and the capacity of our visual system to perceive and interpret visual data. This paper describes the use of sonification to interpret brain scans and use sound as a complementary tool to view, analyze, and diagnose. The sonification tool may be used as a method to augment visual brain data display.

### CROSS-REFERENCE TO RELATED APPLICATIONS

This application claims priority from U.S. Provisional Application 61/916006, filed Dec. 13, 2013, and is incorporated herein by reference in its entirety.

### STATEMENT OF GOVERNMENT INTEREST

The United States Government may have rights in the invention described herein pursuant to NIH/NCATS UL1 TR000038.

### FIELD OF THE INVENTION

The present invention generally relates to the presentation of information. Specifically, the present invention relates to sonification of imaging data for display.

### BACKGROUND OF THE INVENTION

Modern medicine relies heavily on human perception as a means to detect and monitor disease. Techniques based on (1) verbal communication and (2) physical exam evolved over hundreds of years, long before the development of effective therapies.

A variety of human sensory systems and cognitive pathways are put to use by the physician when evaluating a patient. Verbal communication involves not only listening to words spoken but also an assessment of more subtle clues including a patient's tone of voice and gestures. Physical exam requires the clinician to visually inspect, listen to and touch the patient. Even a doctor's sense of smell can aid in diagnosis. It is clear from the above that physicians employ all the major physical senses in their pursuit of information regarding their patients.

A broad category of diagnostic testing, which has revolutionized diagnosis and management of disease, is medical imaging. A variety of techniques based on x-rays, mechanical vibration, fluorescence, rotation of atoms and radioactive decay have been developed and produce multi-dimensional and time-varying arrays of spatial information.

Current methodologies used to “perceive” and interpret medical image data are largely based on the human visual system, in some cases enhanced by use of simple graphs or tables depicting numeric data. Such visual and traditional quantitative analysis methods have led to great advancements in nuclear medicine, radiology and other fields.

The diagnosis of medical conditions has been transformed with the advancement of medical imagining techniques, including X-rays, magnetic resonance imaging (MRI), and positron emission tomography (PET). These techniques provide physicians with multi-dimensional and time-varying datasets that continue to increase in precision and resolution. Until recently, the data acquired by these imagining techniques has been primarily presented with visual displays, using visual analysis as the principal method of evaluation and diagnosis.

Despite these innovations, many limitations remain with respect to the medical community's ability to perceive and analyze the vast amounts of data now being generated by CT scanners, PET scanners, MRI machines and newer combined devices (including PET/CT and PET/MRI). Diagnostic accuracy is higher than ever, but clinicians still are unable to detect certain conditions when the information provided by visual analysis or basic quantization does not uncover perceptible differences between disease and health. Quantitative analysis techniques for examining medical image data represent a significant step forward beyond the traditional visual processing system of the human brain.

### SUMMARY OF THE INVENTION

One embodiment of the invention relates to a method for sonifying image data, comprising: receiving imaging data regarding a region of interest and sonifying the imaging data. The sonification comprises defining a sonification path and mapping the imaging data to at least one audio parameter using the defined sonification path, to define a sonified audio signal of the image data.

Yet another embodiment relates to a method of detecting brain pathology comprising: receiving imaging data of a brain; normalizing the imaging data; selecting a subset of the imaging data; segmenting the subset of the imaging data into at least three lobes of interest; assigning a wave oscillator to each of the three lobes of interest; and sonifying the imaging data. Sonification comprises defining a sonification path, mapping the imaging data to at least one audio parameter using the defined sonification path, to define a sonified audio signal of the image data. The sonified audio signal is played.

Yet another embodiment relates to a computer-implemented machine for medical image sonification. A processor is provided. Tangible computer-readable medium operatively connected to the processor and including computer code configured is provided. The code is configured for: receiving medical imaging data regarding a region of interest and sonifying the medical imaging data. Sonification comprises: defining a sonification path and mapping the medical imaging data to at least one audio parameter using the defined sonification path, to define a sonified audio signal of the medical image data.

Additional features, advantages, and embodiments of the present disclosure may be set forth from consideration of the following detailed description, drawings, and claims. Moreover, it is to be understood that both the foregoing summary of the present disclosure and the following detailed description are exemplary and intended to provide further explanation without further limiting the scope of the present disclosure claimed.

### BRIEF DESCRIPTION OF THE DRAWINGS

The foregoing and other objects, aspects, features, and advantages of the disclosure will become more apparent and better understood by referring to the following description taken in conjunction with the accompanying drawings, in which:

FIG. 1 Schematic of a sonification tool.

FIG. 2A-C Sonification paths: FIG. 2A left-to-right; FIG. 2B top-to-bottom; and FIG. 2C all data.

FIG. 3A-B Representation of vertical FIG. 3A and horizontal FIG. 3B HRTF spatialization.

FIG. 4A-D Images of brain scans (left column) and spectrograms of sonifications (right column) of healthy brains (FIGS. 4A and 4B), and unhealthy brains with Alzheimer's dementia FIG. 4C and frontotemporal dementia FIG. 4D.

FIG. 5 Lobe segmentation of lateral slice.

FIG. 6 Screenshot of an interface for interactive sonification playback and recording.

FIG. 7 illustrates a computer system for use with certain implementations.

### DETAILED DESCRIPTION OF THE PREFERRED EMBODIMENTS

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and made part of this disclosure.

Sonification is defined, or at least accepted as “the transformation of data relations into perceived relations in an acoustic signal for the purposes of facilitating communication or interpretation.” Sonification involves the translation and integration of quantitative data through mapping to a sound model, and enables recognition of patterns in data by their auditory signatures.

Information presentation is critical for both research and education. Scientists frequently rely on highly developed visualization techniques for their own understanding, as well as an aid in presenting material to lay audiences. Effective use of sound hinges on perceptual understanding, and what types of tasks we use the eyes and ears for. Visualizations are strongly synoptic, that is, an entire image can be seen at once. The eyes provide summary information of features such as shape, size, and texture. Many organizing principles of visual cognition also apply to auditory perception. Like the eyes, the ears create auditory gestalts that aid understanding the nature of events, and make estimates when presented with incomplete information.

Two areas that could benefit significantly from improved display and analysis are diagnostic accuracy and inter-observer variability. Although diagnostic accuracy is higher than ever, clinicians still have difficulty detecting certain conditions when the visual analysis of the information provided leads to imperceptible differences between health and disease.

One important difference between visualization and sonification is that sonification exists in time. It cannot be “listened to all at once.” Being time-based, the ears give us a strong sense of dynamic elements of our environment. Further, the human auditory system is also highly adapted for following multiple streams of information. Thus, sonification is an effective way to display a multitude of signal processing operations simultaneously, with each being represented as a line of counterpoint, a series of chords, or a succession of musical instruments. The auditory system is also extremely adept at pattern recognition, a capability that allows us to recognize melodies in spite of transpositions or variations.

The auditory system is most sensitive to dynamic changes involving periodicities: small changes in pitch or tremolo rate are perceptible to untrained listeners. Beyond this, other dimensions that may be represented in an auditory display include changes in loudness, instrument, stereo position, spectrum, transient time, duration, and distance. Displaying 3D data on visual displays is particularly challenging because of the many dimensions needed to be presented concurrently. Human eyes are limited with their “field of view”, and cannot see behind objects that obstruct the view. Our hearing, however, is omni-directional, and we can hear sounds emanating from around us, and in distance.

In one embodiment, systems and methods are provided for processing imaging data to translate information into an auditory format. It is believed that auditory processing of medical image data will identify patterns associated with disease that cannot be detected by traditional means, such as visual presentation. Various imaging techniques, such as three-dimensional imaging, can be used to generate the imaging data that is then sonified for auditory presentation.

Specifically, in one embodiment, molecular brain imaging detects small molecules through the use of radioactive decay of injected “tracers” (PET imaging). Patterns, when sonified, will emerge from the data and show information that could not be previously detected by visual review and pre-existing computer analysis techniques. These patterns, when heard and processed by the human brain, will allow the medical community to detect diseases that are presently invisible by currently existing methodologies.

Imaging is typically done of a zone of interest. For example, a brain image may focus on a zone of interest within the brain, such as aparticular lobe or area of a lobe, i.t. the lobe of interest. For purposes described further below, the entity being imaged, again as an example the brain, may be divided into regions. The brain, as described in the example below, may be divided into hemispheres or regions such as lobes. For utilizing sonification for identification of a particular pathology, a reference region, such as a reference lobe, unaffected by the pathology may be utilized for comparison to the region of interest, such as a lobe of interest.

Different types of sonification may be utilized, for example direct sonification, parametric sonification, and immersive sonification.

Direct sonification utilizes datasets that are in the form of linear arrays. The linear arrays include a temporal sampling of a parameter of the associated system. Direct sonification correlates each element in the linear dataset to a sample value in an audio waveform. Because audio waveforms can contain thousands of samples per second, very large datasets can be represented by even short time spans. The sample rate of the sonified data can be selected (or varied) to provide different temporal resolutions to the dataset. Direct sonification provides periodicity, which is an auditory feature that human hearing can readily discern.

In parametric sonification different properties of a dataset are mapped to different auditory parameters in a sonified signal. For example, if a linear array of data is used, the sonified signal can be a sine wave and the data values can be mapped to frequency of the sine wave over time. Parametric sonification is particularly useful in auditory representation of multi-dimensional datasets. Auditory parameters that can be utilized include, but are not limited to, pitch, amplitude, envelope, and timbre.

Further, immersive sonification is a method of sonifying data in such a way as to place the viewer/listener of this data inside of an immersive 3D environment so that she may be able to navigate through the environment. The sonified data is rendered constantly. However, the listener will be closer/farther to a region of the data depending on their virtual location in the immersive data environment. For example, one can think of an immersive sonification of 3D brain data as putting the listener inside of the scanned brain. An immersive environment is created through the use of spatial sound in order to present a spatially distributed sonification.

In order to appreciate immersive sonification and its applications, it is important to understand the complexity that a human is able to discern. Spatial hearing refers to the ability of human listeners to judge the direction and distance of environmental sound sources. To determine the direction of a sound, the auditory system relies on various physical cues. Sound waves emanating from a source travel in all directions away from the source. Some waves travel to the listener using the most direct path (direct sound) while others reflect off walls and objects before reaching the listener's ears (indirect sound). The direct sound carries information about the location of the source relative to the listener. Indirect sound informs the listener about the space, and the relation of the source location to that space.

The Duplex Theory of Sound Localization states the two primary cues used in sound localization are time and level differences between the two ears. Because of the ears' spatial disparity and the mass between them, they each receive a different version of the arriving sound. The ear that is closest to the sound (ipsilateral ear) will receive the sound earlier and at a greater intensity or level than the ear farther away from the source (contralateral ear). The differences in time of arrival and in level are referred to as the Interaural Time Difference (ITD) and the Interaural Level Difference (ILD) respectively.

Although the ITD and ILD cues are good indicators for determining the location of sources along the interaural axis, they provide an insufficient basis for judging whether a sound is located above, below, in front or in back. For sources located at an equal distance on a conical surface extending from the listener's ear, ITD and ILD cues are virtually identical producing what is referred to as the “cones of confusion”. Along a cone of confusion, where the ITD and ILD cues along a cone of confusion for a source located in the front, back, up and down are equivalent, a listener can have difficulty determining the difference in location, which can lead to front/back or up/down confusion.

There is an additional acoustic cue that helps to resolve the position along a cone of confusion. Before reaching the listener's ears, the acoustic waves emitted by a source are filtered by the interaction with the listener's head, torso and the pinnae (outer ear), resulting in a directionally dependent spectral coloration of the sound. This systematic “distortion” of a sound's spectral composition acts as a unique fingerprint defining the location of a source. The auditory system uses this mapping between spectral coloration and physical location to disambiguate the points along a cone of confusion, leading to a more accurate localization of a sound source. The composite of the ITD, ILD and the spectral coloration characteristics are captured in Head-Related Transfer Functions (HRTF).

### Sonification Tool

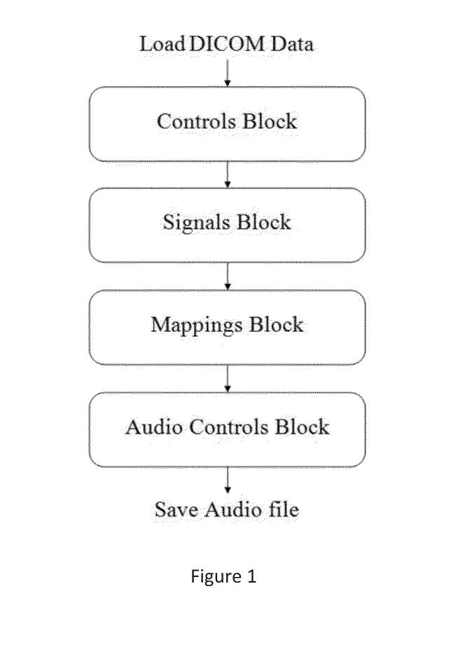

One embodiment provides a sonification tool to sonify imaging data, in particular for recognition and use by the human auditory system. A sonification tool, for example one developed in Matlab® technical computing software. provides a graphical interface through which a user can load, manipulate, and sonify imaging data. In one embodiment, the imaging data is Digital Imaging and Communications in Medicine (DICOM) data—a standard format for viewing and distributing medical imaging data. The data will be referred to as DICOM data for ease of reference, but it should be appreciated that different formats or types of data can be utilized. The data and signal flow are illustrated in FIG. 1.

While a sonification tool having various operational structures, it may be developed with a module design to accommodate different imaging technologies for providing the data and analyzing the imaging data for various purposes such as modules specific to identifying particular pathologies. The following four modules have been designed to map the data to sound: control path, sound synthesis, mappings, and audio controls. DICOM data is read into the sonification tool, and data manipulation is performed in the controls module. After passing through the control path module, which specifies the data path the sonification will follow, the sound synthesis module defines the details of the sound synthesis mechanism and passes to the mappings module. Sound is generated using these parameters and the resulting audio is played using the audio controls module.

In one embodiment, the imaging data is provided in slices, such as the lateral slice of a brain scan. The data values of each lateral slice are normalized such that the voxel with the maximum data value is scaled to 1 and the voxel with the minimum data value is scaled to 0.

### Control Module

The Control module reads in and prepares the DICOM data to be sonified. This module has five main functionalities: data read, data selection and zoom, sonification duration, and data adjustment.

The sonification tool loads DICOM-formatted files. A 3D matrix of the scan data is constructed and used for the sonification and the basic visual display provided. In one embodiment, the 3D matrix is a direct representation of the 3D DICOM data obtained from a PET scan. A single frame of data may be selected for sonification, a subsection of one frame, or a full three-dimensional scan. Using HRTF processing, the data selected is spatialized, a process by which directly correlates the location of each data voxel to an apparent sound location heard by the listener.

Once the data is loaded, it may be adjusted. Transposing the DICOM data values that are suitable for auditory presentations requires that all data be easily re-scalable. Additional controls of range, shift and volume allow the mapping ranges to be adjusted. Range control is performed by extending or contracting the range of the data (e.g. extending the data range from 1-100 to 1-200). Data shift moves the data values to a different range—transposing pitches to higher or lower part of the musical scale (i.e. like shifting to higher or lower notes on a piano). The range adjusts the data range. It allows a lesser or greater differentiation between the largest and smallest values in the data. The adjustments are done by the user in order to facilitate a better perception of the resulting sound.

### Sonification Path Module

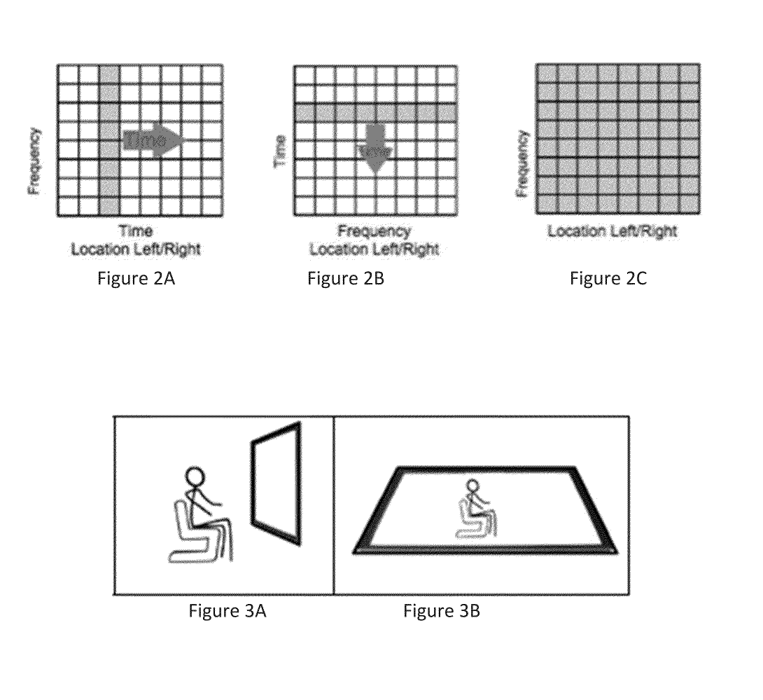

The sonification of the data stored in a matrix can be done by sonifying one data voxel at a time, by row, by column, or sonifying the full 2D or 3D matrix simultaneously. The Sonification Path module allows the user to define the exact path through which to scan the selected data. The sonification tool contains three preset paths: from left to right, top to bottom, or all data simultaneously, as seen in FIG. 2. If the left-to-right path is selected, each data column is sonified and played concurrently, followed by the next column of data. If the right-to-left path is selected, each row is sonified, and rows are played concurrently. If simultaneous is chosen, effectively the path is removed: the sum of each row is taken, resulting in a single column, with each value representing the total values of the corresponding row. We refer to these three mapping trajectories as conventional paths.

In addition to the three conventional paths, we also allow sonification along a split-path. The selected data frame is split along a specified line (split line) and sonified as two halves. The split-line is specified by the Cartesian x and y coordinates of any two points on the line. The motivation behind the split path is to facilitate detection of asymmetries between the two halves of the data, which correspond to the two brain hemispheres.

There are two types of split paths. The first is a hard left/right, where the selected data is split into two halves, the left side is sonified and played to the left ear, and the right side is sonified and played to the right ear. The second type is the difference scan, where the mirror of the right side of the data is subtracted from the left side. This can be thought of as folding the halves onto each other and taking a difference between the two halves. Resulting asymmetries between the two sides of the data are the only audio signals that are audible, since symmetric data is cancelled out. In both types of split paths, the sonifications generated are based on the mappings and scan paths specified, as described above.

### Sound Synthesis Module

The Sound Synthesis module specifies the base signals to use for the sonification. Although examples of signal types are described herein additional signal types can be used as well. Any combination of signals contained in this module can be used simultaneously in the sonification. In one implementation, the sonification can use band-passed noise, triangle wave tone, or a plucked string model, or a combination thereof. For example, when band-passed noise is selected, a noise signal is used for the sonification that is band-passed at the centre frequency that corresponds to a data value.

The parameters of these signals, namely amplitude, frequency, spatial location, and time of occurrence, are governed by the data to be sonified. The manner of mappings is specified in the Mappings block (next section). Any number of base signals can be simultaneously selected for sonification, and doing so will layer the signals on top of each other.

### Mapping Module

The Mappings module defines how the image data is mapped to different parameters of the audio. As noted above, there are several ways in which the image data can be mapped or correlated to audio. Three different mapping techniques are described herein as illustrative examples: amplitude mapping, frequency mapping, and spatialization.

In the case of amplitude mapping, each voxel's value is mapped to the physical amplitude of the audio signal corresponding to that voxel. The greater the data value, the greater the amplitude of the audio signal resulting in a louder sound. Therefore, highly active regions of the data result in louder regions. In an alternative embodiment, for example where a lack of activity reflected in an image is the “state” being looked for, each voxel's value is mapped inversely with intensity such that low activity results in the most intense sound. A pre-determined scale may be used to set the amplitude at a certain level for any intensity above a determined value.

When the mapping is performed, different frequencies are assigned to different voxels based on their location in the image. I n on e embodiment, frequencies are distributed between 500 Hz and 5 kHz. How the frequencies are distributed depends on the sonification paths. For Path 1 (left to right), frequency varies from top to bottom, with the highest frequency assigned to the topmost rows in the data (FIG. 2A). For Path 2 (top to bottom), frequency varies from left to right, with highest frequency assigned to the rightmost column (FIG. 2B). Path 3 (all data) follows the same frequency assignment as Path 1 (FIG. 2C). While Paths 1 and 2 present each row or column sequentially, Path 3 renders an integrated spectrum of the entire image, since the same sets of frequencies for every column are presented simultaneously. A total sum of values is taken for each row/frequency, and this sum controls the amplitude of each frequency component.

In the case of frequency mapping, voxels are assigned frequency based on their intensity, with all amplitude values being kept at a constant level. In one embodiment, the assignable frequencies are quantized to integer multiples of 500 Hz to a maximum of 5 kHz, so all generated audio contains only harmonic content. In an alternative embodiment, the relationship between frequency and intensity may be inverse or frequency may have a different property of the image data associated therewith.

The motivation behind performing frequency mapping is to judge the intensity distribution of a particular dataset through timbre. Datasets containing more high-intensity voxels will have more high-frequency harmonic content. This may in some cases result in a harsh timbre, if there is a large range of values being presented and many frequencies are sounding, or a ringing, if most values are high, and thus each voxel is rendering the same frequency. Hence, through frequency mapping, the user can gain a quick idea of the composite intensity at each step along the selected scan path.

The Spatialization by the mapping module distributes sonified sound spatially around the listener. Assuming users would employ off-the-shelf headphones of reasonable fidelity, a great range of apparent stereo locations may be synthesized. Spreading the sound around the listener can result in a better differentiation of sonified regions, and thus a better distinction of features pertaining to sections of the data.

In one embodiment, various spatial mapping methods may be employed by the sonification tool, including, for example: intensity panning, panning using ILD and ITD, vertical spatialization, horizontal spatialization, and full 3D spatialization. Spatialization methods of the sonifications may be presented over headphones using binaural processing, or through loudspeakers.

The intensity panning method uses interaural intensity differences between the two ears to pan sounds from left to right. When the sounds sent to the left and right ears are of equal level, the virtual auditory image appears to be located in the center. As the sound level of one of the ears increases, the location of the source appears to be originating from the side with the greater sound level. Intensity panning is effective for creating changes along the horizontal plane, but not the vertical plane. Using the intensity panning method, the audio corresponding to each voxel is panned to an apparent position based on the Cartesian x-coordinate of the voxel. Voxels that are in the center of the image will be panned and perceived to be coming from the center, those that are on the right side of the image will be panned to the right, and so on. A left-to-right image path will result in the sound containing all the vertical image data moving from left to right, while in a top-to-bottom image path the sound constantly surrounds the listener from left to right, and the vertical data is presented consecutively.

Vertical HRTF spatialization utilizes head-related transfer functions to map the selected dataset onto a two-dimensional (up-down, left-right) vertical aural image space directly in front of the user. The spatialized audio correlates with the spatial distribution of the visual image. For example, data on the top left corner of the image is sonified and perceived to be coming from a high elevation, the left data that is in the middle of the image is presented at ear level, and data in the lower part of the image is spatialized below ear level (FIG. 3A).

Horizontal HRTF Spatialization utilizes head-related transfer functions to map the selected dataset onto a two-dimensional horizontal aural image space (front-back, left-right) that places the user in the center. Effectively, the image is laid flat on the horizontal plane, and the listener is placed in the center of the image. The audio corresponding to the sonified dataset is spatially placed all around the user on the horizontal plane (FIG. 3B). For example, data that is in the upper left corner of the image would be sonified and presented in the left front of the audio image.

The full 3D spatialization method can be used for data that is three-dimensional. This would be a VR version of the data, with a series of horizontal or vertical scans all active simultaneously. HRTFs and distance mapping are used to position the data points around the listener—front, back, left, right, up, down. In this case, all the data can be sonified concurrently and spatialized to reflect the position of each data point relative to the listener. The listener can place herself in the middle of the data, or listen to the data from another perspective. For example, the listener can be center of a 3D brain scan and listen to all the data in the scan concurrently. By selecting different listening locations, the listener can effectively “walk through” the data in a fully immersive manner.

### Audio Control Module

The Audio Control module manages the sonification process and handles the generated audio files. Once the relevant sonification and data parameters are set, the sonification is performed, and the rendered sonification can be played or stored to disc.

There is an additional A/B playback functionality, which facilitates the serial playback of two different sonifications as an A/B comparison. This is useful when comparing two sets of data that may have been taken at different points in time, or comparing two cross-sections of the same dataset in order to perceive the difference between the two. This capability for A/B comparison through sonification function is particularly important since the perceptual auditory system is more acute than the visual system at detecting temporal, spectral, and spatial changes. When small changes in data occur, they may not be immediately noticeable on the visual display, but may be more easily observed using sonification. To enable the A/B comparison, two datasets are loaded and selected for sonification. After each dataset is sonified, the listener can listener to the two sonifications in succession in an A-B-A-B-A-B (etc.) manner. In effect, one of the two datasets becomes a reference. The presence of sonic differences between the two datasets becomes audible.

### EXAMPLES

### Brain Scans

Brains scans contain complex, highly variable data and present a challenge to the interpreting physician. Imaging experts spend years learning to properly read such studies, yet detection of subtle disease remains difficult. Compounding matters, many disease processes remain invisible even to the best observers, either due to lack of meaningful information or undiscovered means by which to identify the relevant data. Visual quantitative techniques have improved matters but there is room for further improvement. It is suspected that as-yet-undiscovered information exists within these images and has diagnostic and therapeutic relevance.

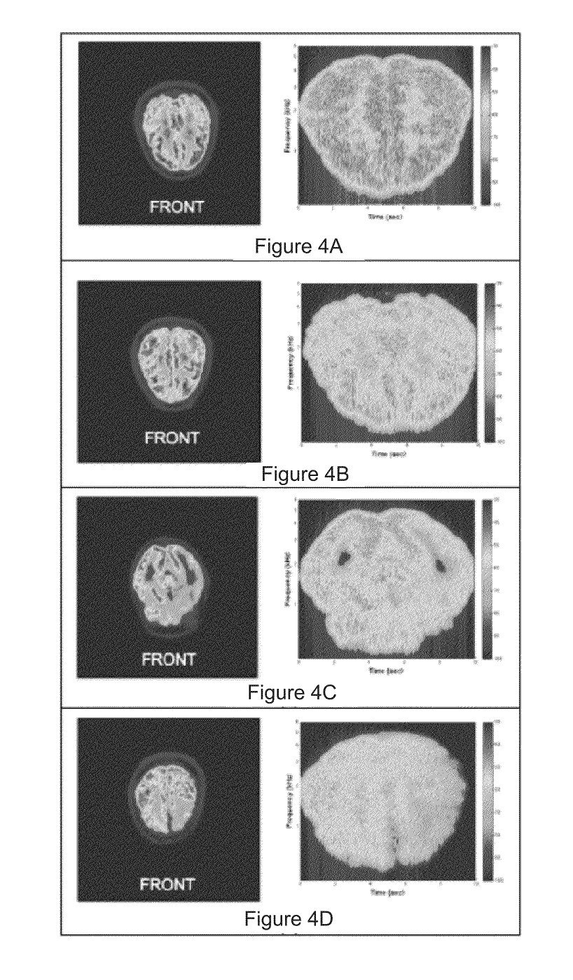

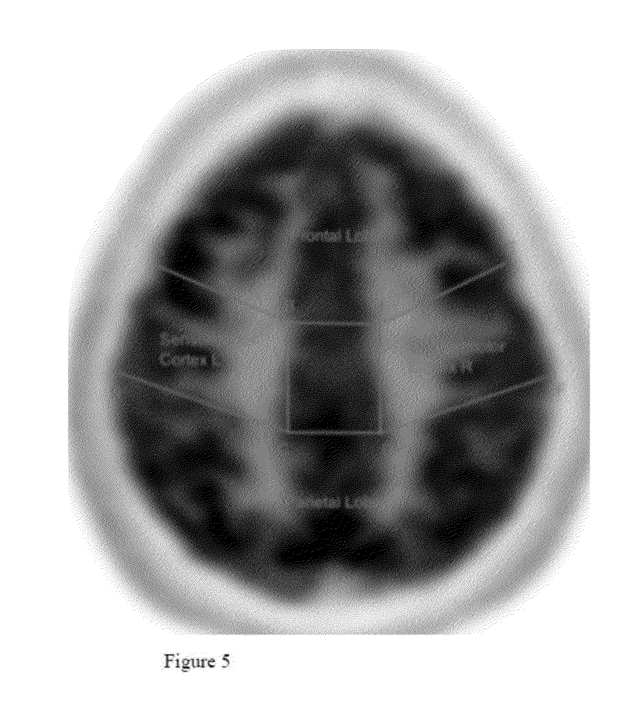

FIG. 4A-D contains four examples of the spectrograms of sonifications, utilized for illustration purposes to show the correlation between the PET scans and their sonifications, that were created from normal and abnormal brain data. The left side of the figure contains single slices from three-dimensional PET scans depicting sugar utilization in the brain. On the right side are the spectrograms of the sonifications. In these examples, the x-axis of the spectrogram represents time, while the y-axis shows the frequencies from low (bottom part of the graph) to hi (top part of the graph). The intensity of the color represents the amplitude of the spectral content at each point in time—with blue indicating low amplitude, and red high amplitude.

The top two examples FIG. 4A and FIG. 4B—are of healthy brains exhibiting a homogenous and symmetric pattern of sugar metabolism. As can be seen in the spectrograms (and heard in the sonifications), the symmetry of the spectral content as well as the full bandwidth reflects normal glucose uptake in the brain. Conversely, in FIG. 4C, there is asymmetric and decreased signal intensity in the brain of a patient with Alzheimer's dementia. A lack of low frequency content is heard in the sonification, and is visible in the second part of the spectrogram, starting at approximately 13 seconds. This region on the spectrogram corresponds to the most severely diseased portion of the brain, resulting in an audible hole in the frequency spectrum. Likewise in FIG. 4D, an image of a brain scan of a patient with frontotemporal dementia is presented and demonstrates a lack of low frequency spectral content corresponding to the frontal lobes of the brain. This leads to an unusual sonic representation of that hemisphere.

During a cursory comparison of normal and abnormal brain scans, it was immediately apparent that there was much more scattered activity with the dementia scans than with the normal scans, and there was a certain sonic quality that might be described as more “strident” in the central area of the scan. By zooming in on the central area of greatest activity, it became apparent that the dementia scans consisted of activity farther forward in the brain, due to the later starting time of audible activity in the L-R scan.

As understanding of MRI, PET and other imaging technology increases, novel means of data presentation and analysis can only be welcome. Imaging scientists are constantly searching for new ways to examine their massive data archives, and sonification is an intriguing line of inquiry that complements other investigations in image analysis. Non-invasive imaging coupled with new perceptual methods such as sonification hold great promise for a future in which society can better detect and treat disease. Such advancements will be welcomed by doctors and, most importantly, patients.

### Dementia Case Study Example

One example utilized the sonification tool and associated methods where the brain is segmented into three regions, and each region is mapped to a different frequency. The interaction of the tones of different frequencies results in beating patterns, which are easily perceived by the human ear. The different beating patterns that can be created, can be illustrated mathematically through additive synthesis, where frequencies are added point by point. The basics of additive synthesis tell us that, when two frequencies are added together, an oscillating amplitude envelope is created at a rate that is the difference between those two frequencies; otherwise known as a beat frequency or beat envelope. If the frequencies are very close together, the psychoacoustic phenomenon is understood more on a tonal level, i.e. our brain would interpret these two distinct frequencies as one frequency that is an average of the two, along with the beat envelope around that frequency. If the frequencies are further apart, the psychoacoustic phenomenon is more temporal, as our brains interpret them as distinct frequencies peaking at different times, again with the beat envelope around these frequencies. Adding a third frequency adds two more beat envelopes, and more possibilities for tonal and rhythmic complexities. Thus each brain scan has a unique rhythmic pattern, a signature sound. This research exploits the ear's sensitivity to complex rhythms through the psychoacoustic phenomenon of beating frequencies.

The sonification method presented here has its basis in the field of medical imaging informatics. In the case of diagnosing different forms of dementia, the metabolic activity in different regions of the brain needs to be measured and compared. Alzheimer's Disease (AD) is characterized by decreased metabolic activity (hypometabolism) in the parietal and temporal lobes of the brain. More severe cases of AD also present hypometabolism in the frontal lobe of the brain. Hence, the diagnosis of AD in patients is performed by comparing metabolic activity of the parietal, temporal, and frontal lobes to the metabolic activity of other lobes that are not generally affected by Alzheimer's disease, such as the sensorimotor cortex. PET scans provide a 3-dimensional measurement of metabolic activity in different regions of the brain. This sonification method aims to aurally display the metabolic activity of these lobes of interest.

The datasets utilized for sonification consisted of 32 de-identified PET/CT scans of human brains diagnosed with varying stages of Alzheimer's disease, obtained from the Radiology Department of New York University Langone Medical Center. These 32 brains scans consisted of 8 brain scans in each of the four categories of diagnosis of AD; Normal, Mild AD, Moderate AD, and Severe AD.

All datasets utilized for sonification were spatially warped to a standard brain model, and hence were all spatially consistent. Spatial normalization is considered a necessary step in conducting statistical analysis across several brain datasets. This process was done using the medical imaging software MIM®, a data visualization tool for PET scan data. In addition to pure visualization tools, MIM Software also contains statistical analysis tools that are capable of segmenting the brain into its various lobes and providing statistical deviations from normalcy based on a standard database of normal brains.

After spatial normalization all dataset pre-processing and lobe segmentation was performed with the sonification tool, our primary data analysis tool for sonification. The sonification tool used in these examples was developed in the C++ programming language.

In the case of each brain, a subset of each dataset was chosen for sonification; the 30th lateral slice (from the top) of the spatially normalized dataset. This particular slice of each spatially normalized dataset passes through representative regions of the frontal lobe, parietal lobe, and sensorimotor cortex.

### Lobe Segmentation

The spatially normalized sub-datasets were then segmented into the three lobes of interest (frontal and parietal) and the reference lobe (sensorimotor cortex). This segmentation was performed with the aid of the MIM software. MIM performs its own automatic lobe segmentation of the spatially normalized brain datasets and provides the segmentation information to the user in the form of the DICOM standard RTSTRUCT file format. These files contain 3-D contours called Regions of Interest. However, for the sake of time, the RTSTRUCT files were not utilized in their entirety for this work. Only the RTSTRUCT's coordinate points, outlining the general boundaries of the lobes of the spatially normalized sub-dataset, were used. These coordinates were set in the sonification tool and the contours were approximated to straight lines connecting the coordinates (FIG. 5).

This approach meant that there remained some irrelevant data points within each lobe's segmentation. First, some data points in the lateral slice lie outside the actual brain area. The second category of irrelevant data points consisted of those that lie within the brain, but are medically irrelevant. The brain can for the most part be divided into white matter and gray matter. The relevant metabolic activity for the diagnosis of AD is that of the grey matter of the brain, and hence the white matter content is medically irrelevant and should not be included as data that is sonified. Both issues manifest as voxels with lower intensities compared to the areas of interest. Hence, both issues were tackled with one generalized solution. All voxels whose intensity fell below a certain threshold were masked from being sonified. This threshold was set to 45% of the maximum allowable intensity, considering the bit-depth of the dataset. Hence, for datasets with a bit-depth of 15 bits, the masking threshold is set to 14745.15 out of a maximum allowable value of 32767.

### Triple-Tone Sonification

In order for a sonification technique to directly target the diagnosis of AD, the level in metabolic activity in each of the lobes of interest and the reference lobe was mapped to an easily perceivable auditory parameter. The sonification technique presented here is a “triple-tone sonification”, which assigns an oscillator to each of the three lobe of the brain to be sonified, specifically each portion of the segmented lobes that fall on the lateral slice. After experimenting with many fundamental waves (including sinewaves, square waves, triangle waves, and others), it was determined that the triangle wave provided the most obviously perceivable sonification for human hearing. The technique can aim at emulating the mental process of a physician during diagnosis, but presenting the results of this process in the aural domain.

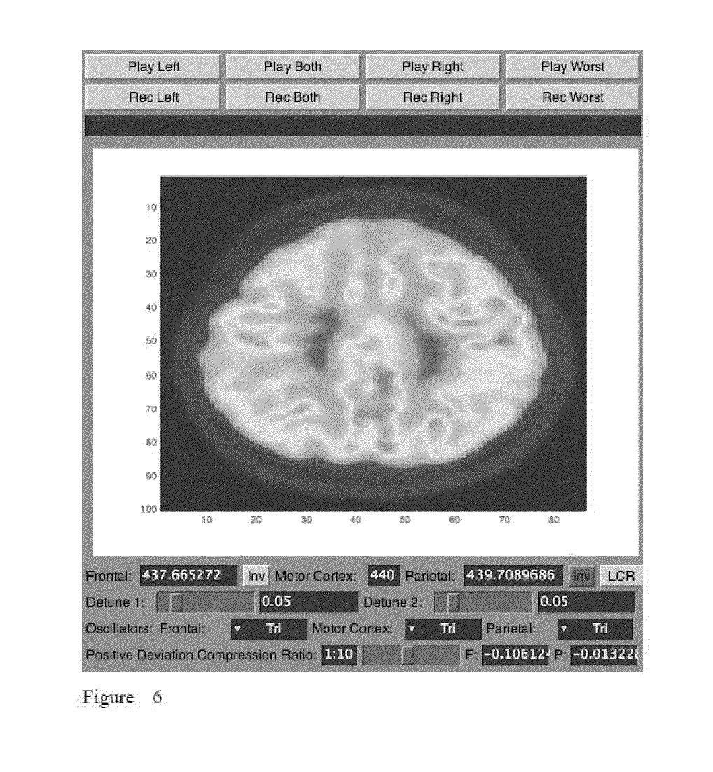

An interactive approach was necessary in order to prototype the technique with versatile functions, to see which auditory parameters best suited the datasets. The sonifications were implemented through the powerful sound synthesis engine and audio programming language, SuperCollider, in the form of a GUI (FIG. 6).

### Frequency Mapping

As described above, the frequencies of these oscillators are mapped. Specifically, they were mapped to the average metabolic activity within each of the three lobes. Hence, differences in metabolic activity between the lobes results in slightly different frequencies of the oscillators. These small differences in oscillation between the three oscillators and triangle wave tones results in beating patterns. The more pronounced the difference between the three frequencies, the faster and more complex the beating pattern becomes. This is directly relevant to the diagnostic method, where higher differences in metabolic activity result in faster beating, more complex rhythms, and eventually splitting of tones.

The frequencies of the tones corresponding to the lobes of the brain under inspection are “detuned” from their default frequency according the deviation of average intensity of the voxels of those lobes from the average intensity of the voxels of the reference lobe(s).

The default frequency used was 440 Hz (A above middle C). The frequency of the frontal lobe was forced to be a positive deviation, and the frequency of the parietal lobe was forced to a negative deviation from the default frequency. Forcing the signs of the deviations of the frontal and parietal lobes ensured that the system would not collapse to two beating tones when there exists abnormalities of equal deviation in both the frontal and parietal lobes. Such a case was undesirable as the complexity of three beating tones was the required artifact to be explored. The goal of triple-tone sonification was to have 3 tones, one associated with each region of interest (lobes in the described example). It could be considered that one lobe is associated with a frequency above the center frequency (440 hz in the example) and one below with the third associated with the center frequency. Preferably, the difference between the three tones is sufficient for human auditory system to distinguish. Further, it should be appreciated while three regions mapped to three tones is described, additional regions with an associated distinguishable tone can be included

### Detune Factor

In one embodiment, a generalizable factor is used to exaggerate the auditory effect for easy of detection/differentiation by a user. In one example, a detune factor was used to control the range of frequency deviations given the range of voxel average deviations. The goal of this parameter was to find different levels of beatings to indicate the varying degrees of AD: For example finding the level of beating that would generate split tones indicating severe cases of AD. A higher detune factor would result in a larger frequency deviation, and hence faster beating, for a set of voxel intensity averages.

One approach to determine the detune factor is to make it a constant across cases to ensure complete standardization of frequency deviations. However, preliminary listening indicated that there was not enough differentiability between AD categories when a constant detune factor was used and only highly experienced listeners could easily perceive and categorize the differences in these beating patterns. In order to test the system on non-expert listeners in the future (e.g. physicians), an exaggeration of the audible effect was created. Hence, in order to exaggerate the detuning effect and improve differentiability, an alternative approach to determine the detune factor would be to generate different detune factors based on some feature of the dataset. To emulate the desired effect, the detune factor was dynamically assigned to different brains according to the table below.

TABLE 1
List of detune factor values

| Brain Type | Detune |
|---|---|
| Normal brain | 0.05 |
| Mild Alzheimer's | 0.10 |
| Moderate Alzheimer's | 0.15 |
| Severe Alzheimer's | 0.20 |

Based on these detune factors, the frequencies of the lobes of interest were determined. The relative deviation of average intensity with respect to the average intensity of the sensorimotor cortex (SMC) was linearly mapped to the relative deviation of the oscillator frequency with respect to the oscillator's base frequency through the detune factor coefficient. This can be represented as follows.

$${f}_{FL} = {f}_{default} * ( 1 + DF * \vert \frac{{av}_{FL} - {av}_{SMC}}{{av}_{SMC}} \vert ) = {f}_{default} * ( 1 + DF * \vert {Δ}_{FL} \vert )$$

$${f}_{PL} = {f}_{default} * ( 1 - DF * \vert \frac{{av}_{PL} - {av}_{SMC}}{{av}_{SMC}} \vert ) = {f}_{default} * ( 1 - DF * \vert {Δ}_{PL} \vert )$$

where f=frequency, av=voxel average, DF=detune factor, FL

=frontal lobe, PL=parietal lobe, SMC=sensorimotor cortex.

### Positive Compression

In most cases of diseased brain datasets, the frontal lobe and parietal lobe averages fell below the average of the SMC. However, in several cases of normal brain datasets, the frontal and parietal lobes possessed averages higher than that of the SMC. This does not represent any abnormality with respect to AD, but manifests itself as an abnormality in the sonifications, as now the frequencies of these lobes would be correspondingly higher or lower than that of the SMC.

In order to differentiate between true abnormality (arising from a lower average value of frontal or parietal lobe activity) and misrepresented abnormality (arising from a higher average of frontal or parietal lobe activity), a “compressor” was applied to all average voxel values before performing frequency mapping. All positive deviations from the average of the SMC were compressed by a 10:1 ratio in all cases presented herein. In the alternative, all values, such as voxel intensity above or below a certain range can be set to a specific hard limit. for example by setting minimum at 0 and anything at the average intensity or above as 1 rather than the maximum intensity as 1.

### Evaluation

### Procedure

The goals of the evaluation were threefold. First, to evaluate the effectiveness of the triple-tone sonification technique in accurate distinguishability between brains of different levels of Alzheimer's disease. Second, to evaluate the intra-reader consistency of diagnosis. Third, to investigate whether a finer gradation of categorization would result in more accurate or consistent results.

In traditional methods of analysis, the diagnosis is typically divided into a categorization of one of four different levels—normal, mild, moderate, and severe. In addition to using the four-step categorization method, we evaluate a finer, seven-step, scale of categorization. The evaluation was divided into two sections: coarse categorization and fine categorization sections. The task of the participant in each section was to categorize the presented sonification into one of four and one of seven categories in the coarse and fine categorizations respectively.

Prior to the test, the participants were presented with training sonifications for the purpose of familiarizing themselves with the nature of each category. Categories 1, 2, 3, and 4 were chosen to correspond to the diagnoses of normal, mild AD, moderate AD, and severe AD respectively. Ten sonifications were generated for each category, resulting in a total of 40 training sonifications.

The training sonifications were generated through the statistical analysis of the 32 brain scans that were used for trials. The frequencies for each training sonification were sampled from a uniform distribution centered on the mean frequency of the corresponding lobe in the corresponding category, with a range of half a standard deviation in either direction. The sonifications used for the training were excluded from the data set presented during the listening experiment.

In the coarse categorization section, the participant was asked to categorize each sonification into one of the four categories (1, 2, 3, or 4), based on the training sonifications. In the fine categorization section, the participant was asked to categorize each sonification into one of the following categories: 1, 1.5, 2, 2.5, 3, 3.5, or 4. Here, the participant was instructed to rate cases that aligned with the training cases to the integer-valued categories, and the cases that may be interpreted as lying between training case categories as the fractional-value categories.

### Datasets

The evaluation used the 32 unique de-identified datasets of patients with varying severity of Alzheimer's disease, obtained from the NYU Langone Medical Center. The collection consisted of 8 datasets in each of the four categories of Alzheimer's disease severity—normal, mild, moderate, and severe. The “ground truth” diagnosis was performed by a medical doctor utilizing visualization and statistical analysis tools provided by MIM software.

In each session, two instances of each dataset's sonification were presented to allow for the testing of intra-reader consistency, resulting in a total of 64 sonifications per sessions. The order of presentation of the sonifications was randomized consistently across participants. The datasets were consistently randomized in each listening session to minimize recall bias.

The subjective listening test was presented to five participants. This control group was chosen to first evaluate the triple-tone sonification technique because of their musically trained ears, providing a validation before proceeding to evaluation with physicians. All medical aspects of this sonification technique were withheld from the participants, and their evaluation of this technique was influenced solely using auditory parameters.

### Results

The accuracy of response against ground truth was computed for each ground truth category for each participant. The accuracy is computed according to:

$$accuracy = \frac{num correct responses}{total num responses}$$

In the case of coarse categorization, a correct response is one where the participant's categorization exactly matches the ground truth of the test case. In the case of fine categorization, a correct response is one that either matches the ground truth, or lies at a distance 0.5 from the ground truth categorization. The results are presented as percentage accuracy for each participant, as well as the mean accuracy for all participants in Table 2 for the coarse categorization, and in Table 3 for the fine categorization.

TABLE 2
Accuracy of participants in coarse categorization
for categories 1, 2, 3 and 4.

| Participan | Cat. | Cat. | Cat. | Cat. 4 | Mean |
|---|---|---|---|---|---|
| P1 | 100% | 88% | 81% | 88% | 89% |
| P2 | 88% | 94% | 38% | 94% | 78% |
| P3 | 94% | 100% | 81% | 100% | 94% |
| P4 | 100% | 94% | 75% | 100% | 92% |
| P5 | 94% | 94% | 44% | 100% | 83% |
| Mean | 95% | 94% | 64% | 96% | 87% |

TABLE 3
Accuracy of participants in fine categorization

| Participan | Cat. | Cat. | Cat. | Cat. 4 | Mean |
|---|---|---|---|---|---|
| P1 | 88% | 88% | 81% | 88% | 86% |
| P2 | 100% | 100% | 56% | 100% | 89% |
| P3 | 100% | 100% | 100% | 100% | 100% |
| P4 | 94% | 100% | 88% | 100% | 95% |
| P5 | 100% | 100% | 69% | 100% | 92% |
| Mean | 96% | 98% | 79% | 98% | 93% |

Each test case was presented to the participant twice in each section to allow for the testing of intra-reader consistency. In the case of coarse categorization, the responses to a pair of duplicate test cases are said to be consistent if both cases were given the same response by the participant. In the case of fine consistency, the responses to a pair of test cases are said to be consistent if both cases were given responses that differ by no more than 0.5. The percentage of the 32 case pairs that were consistent is the consistency percentage. The consistency percentages for each participant in both coarse and fine categorization are presented in Table 4 below.

TABLE 4
Consistency percentage for coarse and fine categorization

| Participan | Coarse | Fine |
|---|---|---|
| P1 | 84% | 88% |
| P2 | 84% | 100% |
| P3 | 94% | 100% |
| P4 | 97% | 88% |
| P5 | 84% | 88% |
| Mean | 89% | 93% |

A side-by-side comparison of participant accuracies is given below for coarse and fine categorization sections.

TABLE 5
Side-by-side comparison of participant accuracy
for coarse vs fine categorization

| Participant | Coarse | Fine |
|---|---|---|
| P1 | 89% | 86% |
| P2 | 78% | 89% |
| P3 | 94% | 100% |
| P4 | 92% | 95% |
| P5 | 83% | 92% |
| Mean | 87% | 93% |

In the case of coarse categorization, mimicking the diagnosis procedure of AD, participants displayed an overall categorization accuracy of 87%. Four out of five participants (all except participant P1) show an increase in categorization accuracy when allowed a finer gradation. Overall accuracy also increased from 87.19% to 92.5% when allowed a finer gradation.

This example illustrates an embodiment of the invention using a triple-tone sonification technique to analyze PET scans of brains with different levels of Alzheimer's dementia. The method was presented and evaluated using subjective testing. Participants involved in the study had no medical experience, but were professional musicians with a trained musical ear.

Results of the evaluation of this sonification method indicate that participants with musically trained ears, are able to categorize the presented sonifications using the triple-tone technique with an average accuracy of 87% using coarse categorization, and an accuracy of 93% when a finer categorization scale was used. This test was comparing the results of the subjective listeners to a baseline diagnosis of a highly experienced medical physician.

The overall accuracy of diagnostic categorization improved with a finer gradation of categorization. This indicates that there exist sonifications generated by this technique that place a brain scan “in between” two categories when evaluated by a listener. In the case of coarse categorization, the in-between scans were perceptually quantized into one of the coarse categories by the participant. In the case of fine categorization, the participant was able to successfully categorize the sonification as an in-between case.

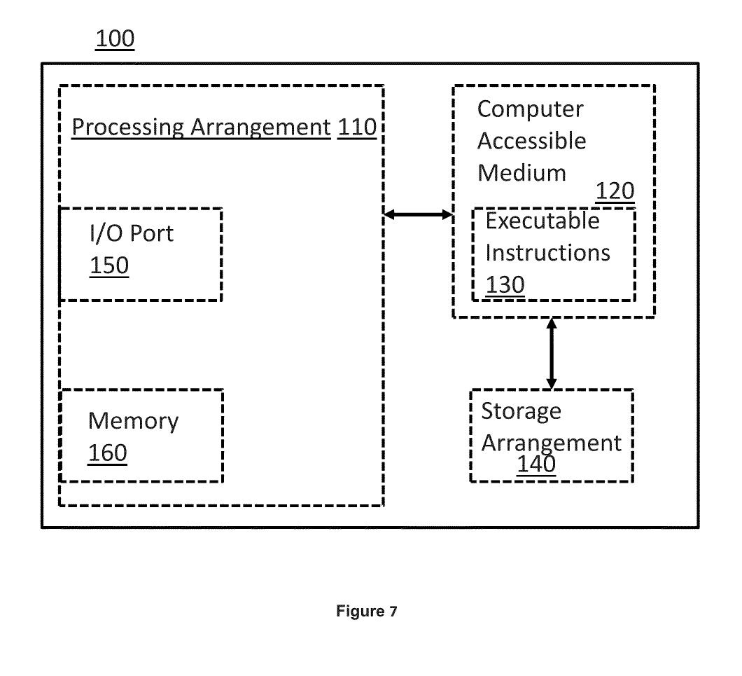

As shown in FIG. 7, e.g., a computer-accessible medium 120 (e.g., as described herein, a storage device such as a hard disk, floppy disk, memory stick, CD-ROM, RAM, ROM, etc., or a collection thereof) can be provided (e.g., in communication with the processing arrangement 110). The computer-accessible medium 120 may be a non-transitory computer-accessible medium. The computer-accessible medium 120 can contain executable instructions 130 thereon. In addition or alternatively, a storage arrangement 140 can be provided separately from the computer-accessible medium 120, which can provide the instructions to the processing arrangement 110 so as to configure the processing arrangement to execute certain exemplary procedures, processes and methods, as described herein, for example. The instructions may include a plurality of sets of instructions. For example, in some implementations, the instructions may include instructions for applying radio frequency energy in a plurality of sequence blocks to a volume, where each of the sequence blocks includes at least a first stage. The instructions may further include instructions for repeating the first stage successively until magnetization at a beginning of each of the sequence blocks is stable, instructions for concatenating a plurality of imaging segments, which correspond to the plurality of sequence blocks, into a single continuous imaging segment, and instructions for encoding at least one relaxation parameter into the single continuous imaging segment.

System 100 may also include a display or output device, an input device such as a key-board, mouse, touch screen or other input device, and may be connected to additional systems via a logical network. Many of the embodiments described herein may be practiced in a networked environment using logical connections to one or more remote computers having processors. Logical connections may include a local area network (LAN) and a wide area network (WAN) that are presented here by way of example and not limitation. Such networking environments are commonplace in office-wide or enterprise-wide computer networks, intranets and the Internet and may use a wide variety of different communication protocols. Those skilled in the art can appreciate that such network computing environments can typically encompass many types of computer system configurations, including personal computers, hand-held devices, multi-processor systems, microprocessor-based or programmable consumer electronics, network PCs, minicomputers, mainframe computers, and the like. Embodiments of the invention may also be practiced in distributed computing environments where tasks are performed by local and remote processing devices that are linked (either by hardwired links, wireless links, or by a combination of hardwired or wireless links) through a communications network. In a distributed computing environment, program modules may be located in both local and remote memory storage devices.

Various embodiments are described in the general context of method steps, which may be implemented in one embodiment by a program product including computer-executable instructions, such as program code, executed by computers in networked environments. Generally, program modules include routines, programs, objects, components, data structures, etc. that perform particular tasks or implement particular abstract data types. Computer-executable instructions, associated data structures, and program modules represent examples of program code for executing steps of the methods disclosed herein. The particular sequence of such executable instructions or associated data structures represents examples of corresponding acts for implementing the functions described in such steps.

Software and web implementations of the present invention could be accomplished with standard programming techniques with rule based logic and other logic to accomplish the various database searching steps, correlation steps, comparison steps and decision steps. It should also be noted that the words “component” and “module,” as used herein and in the claims, are intended to encompass implementations using one or more lines of software code, and/or hardware implementations, and/or equipment for receiving manual inputs.

With respect to the use of substantially any plural and/or singular terms herein, those having skill in the art can translate from the plural to the singular and/or from the singular to the plural as is appropriate to the context and/or application. The various singular/plural permutations may be expressly set forth herein for the sake of clarity.

The foregoing description of illustrative embodiments has been presented for purposes of illustration and of description. It is not intended to be exhaustive or limiting with respect to the precise form disclosed, and modifications and variations are possible in light of the above teachings or may be acquired from practice of the disclosed embodiments. Therefore, the above embodiments should not be taken as limiting the scope of the invention.

What is claimed is:

1. A method for sonifying image data, comprising:
receiving imaging data regarding a region of interest;
sonifying the imaging data, where sonification comprises:
defining a sonification path;
mapping the imaging data to at least one audio parameter using the defined sonification path, to define a sonified audio signal of the image data.

2. The method of claim 1, wherein the sonified signal contains changes in one or more of pitch tremolo rate, loudness, instrument, stereo position, spectrum, transient time, duration, or distance.

3. The method of claim 1, wherein sonifying the image comprises selecting a sonification technique from the group consisting of direct sonification, parametric sonification, and immersive sonification.

4. The method of claim 3, wherein the sonification technique is parametric sonification and further wherein at least one property of the imaging data is mapped to the at least one audio parameter and wherein the audio parameter is selected from the group consisting of pitch, amplitude, envelope, and timbre.

5. The method of claim 3, wherein the sonification technique is immersive sonification further wherein mapping the imaging data comprises mapping between spectral coloration and physical location to disambiguate the points along a cone of confusion.

6. The method of claim 1, wherein imaging data is associated with a slice of a brain.

7. The method of claim 6, wherein the imaging data comprises voxels having values associated with each slice and further wherein the values are normalized such that a voxel with the maximum data value is scaled to 1 and a voxel with the minimum data value is scaled to 0.

8. The method of claim 1, wherein the sonification path is selected from the group consisting of one data voxel at a time, by row, by column, and sonifying the imaging data simultaneously.

9. The method of claim 1, wherein the sonification path is selected from the group consisting of from left to right, top to bottom, all data simultaneously, or split-path

10. The method of claim 9, wherein the sonification path is split-path and further wherein the imaging data is split along a specified line and sonified as two halves.

11. The method of claim 10, wherein the split-path is a hard left/right split path.

12. The method of claim 1, wherein the at least one audio parameter consists of amplitude, frequency, spatial location, and time of occurrence.

13. The method of claim 1, wherein the mapping consists of a technique selected from amplitude mapping, frequency mapping, and spatialization.

14. The method of claim 15, wherein mapping consist of amplitude mapping and wherein amplitude mapping, each voxel's value is mapped to a sound's amplitude.

15. The method of claim 1, wherein the imaging data is divided into subsets corresponding to a plurality of regions.

16. The method of claim 17, wherein each of the plurality of regions is sonified utilizing a different frequency.

17. The method of claim 1, further comprising applying a compression or expansion ratio to all positive deviations within the imaging data prior to mapping.

18. A method of detecting brain pathology comprising:
receiving imaging data of a brain;
normalizing the imaging data;
selecting a subset of the imaging data;
segmenting the subset of the imaging data into at least three lobes of interest;
assigning a wave oscillator to each of the three lobes of interest;
sonifying the imaging data, where sonification comprises:
defining a sonification path;
mapping the imaging data to at least one audio parameter using the defined sonification path, to define a sonified audio signal of the image data;
playing the sonified audio signal.

19. A computer-implemented machine for medical image sonification comprising:
a processor; and
a tangible computer-readable medium operatively connected to the processor and including computer code configured for:
receiving medical imaging data regarding a region of interest;
sonifying the medical imaging data, where sonification comprises:
defining a sonification path;
mapping the medical imaging data to at least one audio parameter using the defined sonification path, to define a sonified audio signal of the medical image data.

20. The computer-implemented machine of claim 19, wherein the medical imaging data is divided into subsets corresponding to a plurality of regions and wherein each of the plurality of regions is sonified utilizing a different frequency.
